(19) United States
(12) Patent Application Publication
(10) Pub. No.: US 2010/0172979 A1
(43) Pub. Date: Jul. 8, 2010

(54) CONTROLLED-RELEASE FORMULATIONS

(76) Inventors: Zhongshui Yu, Bridgewater, NJ (US); Kristin Arnold, Morrisville, PA (US); Siva Rama K. Nutalapati, Princeton, NJ (US)

Correspondence Address: CANTOR COLBURN, LLP, 20 Church Street, 22nd Floor, Hartford, CT, 06103, US

(21) Appl. No.: 12/641,488

(22) Filed: Dec. 18, 2009

Related U.S. Application Data
(60) Provisional application No. 61/140,675, filed on Dec. 24, 2008.

(51) Int. Cl. A61K 9/28; A61K 31/40

(52) U.S. Cl. 424/465; 514/424

(57) ABSTRACT

Disclosed herein are controlled-release formulations containing a core comprising a core active agent (e.g., levetiracetam) and a wax excipient, where the core is substantially coated with an extended-release coating.

### CROSS REFERENCE TO RELATED APPLICATION

This application claims the benefit of U.S. Provisional Application Ser. No. 61/140,675 filed Dec. 24, 2008, which is hereby incorporated by reference in its entirety.

### BACKGROUND

Controlled-release dosage formulations, including sustained-release formulations, provide a variety of benefits to the patient such as reduction in the number of doses per day, increased convenience, reduced occurrences of missed doses, and the chance to achieve controlled blood levels of the active agent.

Levetiracetam, a single enantiomer, (−)-(S)-α-ethyl-2-oxo-1-pyrrolidine is used for adjunctive therapy in treatment of partial onset seizures in patients with or without epilepsy.

An immediate-release tablet containing 250 mg, 500 mg, 750 mg or 1000 mg levetiracetam is currently commercially marketed in the United States. The tablets are administered orally to a patient twice-daily to reach a cumulative daily target of up to 3000 mg per day. Also currently available is a once-daily levetiracetam tablet containing 500 mg or 750 mg levetiracetam.

There remains a need for improved oral pharmaceutical formulations for the controlled release of active agents such as levetiracetam to allow for reduced incidents of administration, specifically single daily dose administrations. Also needed are dosage formulations having substantially no food effect such that a patient has the convenience of taking the dosage formulation with or without food.

### SUMMARY

In one embodiment, a controlled-release formulation comprises a core comprising levetiracetam or a pharmaceutically acceptable salt, solvate, hydrate, crystalline form, or non-crystalline form thereof, and a wax excipient; and an extended-release coating substantially surrounding the core comprising a release-retarding coating material and a plasticizer.

In another embodiment, a controlled-release formulation comprises a core consisting essentially of levetiracetam or a pharmaceutically acceptable salt, solvate, hydrate, crystalline form, or non-crystalline form thereof, and a wax excipient; and an extended-release coating substantially surrounding the core comprising a release-retarding coating material and a plasticizer.

In yet another embodiment, a controlled-release formulation comprises a core consisting of levetiracetam or a pharmaceutically acceptable salt, solvate, hydrate, crystalline form, or non-crystalline form thereof, a wax excipient, a glidant, and a lubricant; and an extended-release coating substantially surrounding the core comprising a release-retarding coating material and a plasticizer.

In another embodiment, a controlled-release formulation comprises a core consisting essentially of levetiracetam or a pharmaceutically acceptable salt, solvate, hydrate, crystalline form, or non-crystalline form thereof, and a wax excipient; and an extended-release coating substantially surrounding the core comprising a release-retarding coating material and a plasticizer; wherein the formulation exhibits substantially no food effect.

These and other embodiments, advantages and features of the present invention become clear when detailed description and examples are provided in subsequent sections.

### DETAILED DESCRIPTION

Disclosed herein are controlled-release formulations comprising a core comprising a core active agent and a wax excipient; and an extended-release coating substantially surrounding the core comprising a release-retarding coating material.

Also disclosed herein are formulations comprising a core comprising a core active agent and a wax excipient; and an extended-release coating substantially surrounding the core comprising a release-retarding coating material; wherein the formulation exhibits substantially no food effect. Furthermore, by choosing the appropriate core materials and coating materials, the resulting formulation possesses enough strength to resist rupture or significant damage to the dosage formulation that could result in a compromise of the release properties. It has been found that a core of a particular hardness (“strength”, e.g., about 10 to about 15 kilopascals (kPa)) provides sufficient support for the controlled-release coating to ensure the integrity of the coating when the formulation is ingested with food. It has also been found that a coated wax core where the core is substantially free of a non-wax binder exhibits a surprising resistance to rupture which allows the dosage form to remain intact up to about 80% of the active agent released from the dosage form.

In another embodiment, the formulation comprises a controlled-release portion in the form of a coated core and an immediate-release portion. The active agent present in the controlled-release portion and the immediate-release portion can be the same or different.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”). The endpoints of all ranges directed to the same component or property are inclusive and independently combinable.

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms, and any polymorphs of the compound are contemplated herein. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

“Pharmaceutically acceptable salts” includes derivatives of the active agent, wherein the active agent is modified by making acid or base addition salts thereof, and further refers to pharmaceutically acceptable solvates, including hydrates, crystalline forms, non-crystalline forms, and polymorphs of such salts. Examples of pharmaceutically acceptable salts include, but are not limited to, mineral or organic acid addition salts of basic residues such as amines; alkali or organic addition salts of acidic residues; and the like, and a combination comprising at least one of the foregoing salts. The pharmaceutically acceptable salts include salts and the quaternary ammonium salts of the active agent. For example, acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and a combination comprising at least one of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and a combination comprising at least one of the foregoing salts.

“Levetiracetam” means levetiracetam or a pharmaceutically acceptable levetiracetam salt, including any solvate, hydrate, crystalline form, and non-crystalline form thereof unless otherwise indicated.

“Reference drug” means a levetiracetam product as described in U.S. Federal Food and Drug Administration's New Drug Application No. 022285 approved on Sep. 12, 2008 (500 mg) or Feb. 12, 2009 (750 mg) as provided in the U.S. Federal Food and Drug Administration's Orange Book, Approved Drug Products with Therapeutic Equivalence Evaluations. Keppra XR™ is a levetiracetam oral, extended-release tablet product available in 500 mg and 750 mg strengths. Keppra XR™, 750 mg is the “reference listed drug” under 21 CFR 314.94(a)(3)), i.e., the listed drug identified by FDA as the drug product upon which an applicant relies in seeking approval of its ANDA.

A “dosage form” or “dosage formulation” means a unit of administration of an active agent. Examples of dosage formulations include tablets, capsules, injections, suspensions, liquids, emulsions, creams, ointments, suppositories, inhalable formulations, transdermal formulations, and the like. “Form” and “formulation” are to be used interchangeably unless indicated otherwise.

By “oral dosage form” is meant to include a unit dosage form for oral administration. An oral dosage form may optionally comprise a plurality of subunits such as, for example, microcapsules or microtablets. Multiple subunits may be packaged for administration in a single dose.

By “subunit” is meant to include a composition, mixture, particle, pellet, and the like, that can provide an oral dosage form alone or when combined with other subunits.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

“Food” typically means a solid food or mixed solid/liquid food with sufficient bulk and fat content that it is not rapidly dissolved and absorbed in the stomach. In one embodiment, food means a meal, such as breakfast, lunch or dinner. The terms “taken with food”, “fed” and “non-fasted” are equivalent and are as given by FDA guidelines and criteria. In one embodiment, with food means that the dosage form is administered to a patient between about 30 minutes prior to about 2 hours after eating a meal. In another embodiment, with food means that the dosage form is administered at substantially the same time as the eating the meal.

The terms “without food”, “fasted” and “an empty stomach” are equivalent and are as given by FDA guidelines and criteria. In one embodiment, fasted is means the condition wherein no food is consumed within 1 hour prior to administration of the dosage form or 2 hours after administration of the dosage form. In another embodiment, fasted means the condition wherein no food is consumed within 1 hour prior to administration of the dosage form to 2 hours after administration of the dosage form.

“Substantially no food effect” means that the pharmacokinetics are substantially the same for the oral administration of the formulation under fed conditions (“non-fasting”) when compared to administration under fasting conditions. For example, the comparison between Cₘₐₓ or AUC of a single administration of a formulation under fed conditions to a single administration of the same formulation under fasted conditions results in a percent ratio of Cₘₐₓ or AUC having a 90% confidence interval upper limit of less than or equal to 125% or a lower limit of greater than or equal to 80%. Such information can be based on logarithmic transformed data. Exemplary study considerations can be found in the Federal Drug Administration's (FDA) guidelines and criteria, including “Guidance for Industry, Food-Effect Bioavailability and Fed Bioequivalence Studies” available from the U.S. Department of Health and Human Services (DHHS), Food and Drug Administration (FDA), Center for Drug Evaluation and Research (CDER) December 2002, incorporated herein in its entirety.

A dissolution profile is a plot of the cumulative amount of active agent released from a formulation as a function of time. A dissolution profile can be measured utilizing the Drug Release Test <724>, which incorporates standard test USP 26 or 28 (Test <711>), incorporated herein by reference in its entirety. A profile is characterized by the test conditions selected such as, for example, apparatus type, shaft speed, temperature, volume, and pH of the dissolution medium. More than one dissolution profile may be measured. For example, a first dissolution profile can be measured at a pH level approximating that of the stomach, and a second dissolution profile can be measured at a pH level approximating that of one point in the intestine or several pH levels approximating multiple points in the intestine.

A highly acidic pH may be employed to simulate the stomach and a less acidic to basic pH may be employed to simulate the intestine. By the term “highly acidic pH” is meant a pH of about 1 to about 4. A pH of about 1.2, for example, can be used to simulate the pH of the stomach. By the term “less acidic to basic pH” is meant a pH of greater than about 4 to about 7.5, specifically about 6 to about 7.5. A pH of about 6 to about 7.5, specifically about 6.8, can be used to simulate the pH of the intestine.

By “immediate-release” is meant a conventional or non-modified release in which greater then or equal to about 75% of the active agent is released within two hours of administration, specifically within one hour of administration.

By “controlled-release” is meant a dosage form in which the release of the active agent is controlled or modified over a period of time; controlled-release is not immediate-release. Controlled can mean, for example, extended-, sustained-, delayed- or pulsed-release at a particular time. Alternatively, controlled can mean that the release of the active agent is extended for longer than it would be in an immediate-release dosage form, e.g., at least over several hours.

Dosage formulations can have both immediate-release and controlled-release characteristics, for example, a combination of immediate-release pellets and controlled-release pellets; immediate-release coating and controlled-release core including a tablet core; and the like. The immediate-release portion of a combination dosage form may be referred to as a loading dose.

The core can be in the form of a particle, a pellet, a bead, a tablet, and the like, specifically as a tablet.

“Bioequivalence” means the absence of a significant difference in the rate and extent to which the active agent or surrogate marker for the active agent in pharmaceutical equivalents or pharmaceutical alternatives becomes available at the site of action when administered in an appropriately designed study.

In one embodiment, bioequivalence is any definition thereof as promulgated by the U.S. Food and Drug Administration or any successor agency thereof. In a specific embodiment, bioequivalence is determined according to the Federal Drug Administration's (FDA) guidelines and criteria, including “GUIDANCE FOR INDUSTRY BIOAVAILABILITY AND BIOEQUVALENCE STUDIES FOR ORALLY ADMINISTERED DRUG PRODUCTS—GENERAL CONSIDERATIONS” available from the U.S. Department of Health and Human Services (DHHS), Food and Drug Administration (FDA), Center for Drug Evaluation and Research (CDER) March 2003 Revision 1; and “GUIDANCE FOR INDUSTRY STATISTICAL APPROACHES TO ESTABLISHING BIOEQUIVALENCE” DHHS, FDA, CDER, January 2001, both of which are incorporated herein in their entirety.

In an embodiment, bioequivalence of a composition to a reference drug is determined by an in vivo pharmacokinetic study to determine a pharmacokinetic parameter for the active agent composition. Specifically, bioequivalence can be determined by an in vivo pharmacokinetic study comparing a pharmacokinetic parameter for the two compositions. A pharmacokinetic parameter for the active agent composition or the reference drug can be measured in a single or multiple dose bioequivalence study using a replicate or a nonreplicate design. For example, the pharmacokinetic parameters for active agent composition of the present invention and for a reference drug can be measured in a single dose pharmacokinetic study using a two-period, two-sequence crossover design. Alternately, a four-period, replicate design crossover study may also be used. Single doses of the test composition and reference drug are administered and blood or plasma levels of the active agent are measured over time. Pharmacokinetic parameters characterizing rate and extent of active agent absorption are evaluated statistically.

The area under the plasma concentration-time curve from time zero to the time of measurement of the last quantifiable concentration (AUC₀₋ₜ) and to infinity (AUC_{0-∞}), Cₘₐₓ, and Tₘₐₓ can be determined according to standard techniques. Statistical analysis of pharmacokinetic data is performed on logarithmic transformed data (e.g., AUC₀₋ₜ, AUC_{0-∞}, or Cₘₐₓ data) using analysis of variance (ANOVA).

Under U.S. FDA guidelines, two products (e.g., an inventive levetiracetam formulation and Keppra XR™, 500 or 750 mg) or methods (e.g., dosing under non-fasted versus fasted conditions) are bioequivalent if the 90% Confidence Interval (CI) limits for a ratio of the geometric mean of logarithmic transformed AUC_{0-∞}, AUC₀₋ₜ, and Cₘₐₓ for the two products or two methods are about 0.80 to about 1.25.

In another embodiment, bioequivalence is determined according to the European Medicines Agency (EMEA) document “Note for Guidance on the Investigation of Bioavailability and Bioequivalence”, issued Jul. 26, 2001, available from EMEA.

To show bioequivalency between two compounds or administration conditions pursuant to Europe's EMEA guidelines, the 90% CI limits for a ratio of the geometric mean of logarithmic transformed AUC_{0-∞} and AUC₀₋ₜ for the two products or methods are about 0.80 to about 1.25. The 90% CI limits for a ratio of the geometric mean of logarithmic transformed Cₘₐₓ for the two products or methods can have a wider acceptance range when justified by safety and efficacy considerations. For example the acceptance range can be about 0.70 to about 1.43, specifically about 0.75 to about 1.33, and more specifically about 0.80 to about 1.25.

In one embodiment, in a given experiment, an active agent composition is considered to be bioequivalent to the reference drug if both the Test/Reference ratio for the geometric mean of logarithmic transformed AUC_{0-∞}, AUC₀₋ₜ, or Cₘₐₓ ratio along with its corresponding lower and upper 90% CI limits are within a lower limit of about 0.80 and an upper limit of about 1.25. Thus, for direct comparison between an inventive active agent composition and a reference drug, the pharmacokinetic parameters for the active agent composition and the reference drug can be determined in side-by side in the same pharmacokinetic study.

In some embodiments a single dose bioequivalence study is performed under non-fasted or fasted conditions.

In other embodiments, the single dose bioequivalence study is conducted between the active agent composition and the reference listed drug using the strength specified by the FDA in APPROVED DRUG PRODUCTS WITH THERAPEUTIC EQUIVALENCE EVALUATIONS(ORANGE BOOK).

In some embodiments, an in vivo bioequivalence study is performed to compare all active agent compositions with corresponding strengths of the reference drug (e.g., 500 or 750 mg of the active agent). In other embodiments, an in vivo bioequivalence study is performed only for the active agent composition of the present invention at the strength of the reference listed drug product (e.g., the highest approved strength) and at the other lower or higher strengths, the inventive compositions meet a reference drug dissolution test.

In one embodiment, the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer, wherein the formulation is bioequivalent to a reference drug according to New Drug Application No. 022285 (Keppra XR™, 500 mg or 750 mg) when administered to a patient in a fasted state.

In another embodiment, the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer, wherein the formulation exhibits a ratio of a geometric mean of logarithmic transformed AUC_{0-∞} of the formulation to a geometric mean of logarithmic transformed AUC_{0-∞} of reference drug (New Drug Application No. 022285, Keppra XR™, 500 mg or 750 mg) of about 0.80 to about 1.25 under fasting conditions.

In another embodiment, the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer, wherein the formulation exhibits a ratio of a geometric mean of logarithmic transformed AUC₀₋ₜ of the formulation to a geometric mean of logarithmic transformed AUC₀₋ₜ of reference drug (New Drug Application No. 022285, Keppra XR™, 500 mg or 750 mg) of about 0.80 to about 1.25 under fasting conditions.

In another embodiment, the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer, wherein the formulation exhibits a ratio of a geometric mean of logarithmic transformed Cₘₐₓ of the formulation to a geometric mean of logarithmic transformed Cₘₐₓ of reference drug (New Drug Application No. 022285, Keppra XR™, 500 mg or 750 mg) of about 0.70 to about 1.43 under fasting conditions.

In another embodiment, the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer, wherein the formulation exhibits a ratio of a geometric mean of logarithmic transformed Cₘₐₓ of the formulation to a geometric mean of logarithmic transformed Cₘₐₓ of reference drug (New Drug Application No. 022285, Keppra XR™, 500 mg or 750 mg) of about 0.80 to about 1.25 under fasting conditions.

In one embodiment, the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer; wherein the formulation exhibits substantially no food effect.

In another embodiment, the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer, wherein the formulation when administered to a patient in a non-fasted state is bioequivalent to the formulation when administered to a patient in a fasted state.

In still another embodiment, the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer, wherein the formulation exhibits a ratio of a geometric mean of logarithmic transformed AUC_{0-∞} of the formulation administered in a non-fasted state to a geometric mean of logarithmic transformed AUC_{0-∞} of the formulation administered in a fasted state of about 0.80 to about 1.25.

In one embodiment, the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer, wherein the formulation exhibits a ratio of a geometric mean of logarithmic transformed AUC₀₋ₜ of the formulation administered in a non-fasted state to a geometric mean of logarithmic transformed AUC₀₋ₜ of the formulation administered in a fasted state of about 0.80 to about 1.25.

In an embodiment, the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer, wherein the formulation exhibits a ratio of a geometric mean of logarithmic transformed Cₘₐₓ of the formulation administered in a non-fasted state to a geometric mean of logarithmic transformed geometric mean Cₘₐₓ of the formulation administered in a fasted state of about 0.80 to about 1.25.

The formulations disclosed herein comprise a core comprising an active agent, a wax excipient, and optionally additional core excipients.

The wax excipient for use in the core can be a solid wax at ambient temperature, such as a solid, hydrophobic material (i.e., non-water soluble) or solid hydrophilic material (e.g., polyethylene glycols are water soluble), but specifically a solid, hydrophobic material.

Exemplary wax excipients include wax and wax-like excipients, for example, carnauba wax (from the palm tree Copernicia Cerifera), vegetable wax, fruit wax, microcrystalline wax (“petroleum wax”), bees wax (white or bleached, and yellow), hydrocarbon wax, paraffin wax, cetyl esters wax, non-ionic emulsifying wax, anionic emulsifying wax, candelilla wax, or a combination comprising at least one of the foregoing waxes. Other suitable wax excipients include, for example, fatty alcohols (such as lauryl, myristyl, stearyl, cetyl or specifically cetostearyl alcohol), hydrogenated vegetable oil, hydrogenated castor oil, fatty acids such as stearic acid, fatty acid esters including fatty acid glycerides (mono-, di-, and tri-glycerides), polyethylene glycol (PEG) having a molecular weight of greater than about 3000 number average molecular weight, Mₙ (e.g., PEG 3350, PEG 4000, PEG 4600, PEG 6000, and PEG 8000), or a combination comprising at least one of the foregoing wax excipients. Any combination of wax excipients is also contemplated.

The melting point of the wax excipient is a temperature above ambient temperature, specifically about 30 to about 150° C., more specifically about 75 to about 100° C., and yet more specifically about 75 to about 90° C.

The amount of wax excipient present in the core can be determined based on the particular wax or wax combination chosen and the targeted release profile desired for the resulting formulation. Exemplary amounts of a wax excipient include about 5 to about 60 wt. % based on the total weight of the core excluding the extended-release coating, specifically about 10 to about 50 wt. %, more specifically about 15 to about 40 wt. %, and yet more specifically about 20 to about 30 wt. % based on the total weight of the core excluding the extended-release coating.

In another embodiment, the core may comprise an antiepileptic/anticonvulsant core active agent such as levetiracetam. Exemplary amounts of active agent in the core include about 60 to about 98 wt. % based on the total weight of the core excluding the extended-release coating, specifically about 65 to about 90 wt. %, and more specifically about 70 to about 80 wt. %.

In one embodiment, the active agent is an antiepileptic drug, specifically levetiracetam. The formulation can contain about 250 mg to about 1.5 grams of levetiracetam, specifically about 500 mg to about 1.0 gram, and more specifically about 750 mg per dosage unit. In one embodiment, the formulation is a tablet containing about 500 to about 750 mg of levetiracetam per tablet.

In one embodiment, the levetiracetam formulations disclosed herein comprise a core that is substantially free of a non-wax binder. As used herein, non-wax binders include cellulosic binders (e.g., hydroxypropylmethyl cellulose, hydroxypropyl cellulose, methylcellulose and the like) crosslinked acrylic acid-based polymers, polyvinylpyrrolidone, starch, pregelatinized starch, modified corn starch, polyacryl amide, poly-N-vinyl amide, sodium carboxymethyl cellulose, gelatin, polyethylene oxide, poly propylene glycol, tragacanth, alginic acid and the like; and the term specifically excludes core excipients such as glidants and lubricants as these excipients are not used as binders. As used herein, “substantially free of a non-wax binder” means the core contains less than 1 wt. % non-wax binder, specifically less than 0.5% non-wax binder, and more specifically 0 wt. % non-wax binder based on the total weight of the core.

The core optionally further contains an additional release-retarding material. Additional release-retarding materials include, for example an acrylic polymer, an alkylcellulose including substituted alkylcellulose, shellac, zein, polyvinylpyrrolidine including crosslinked polyvinylpyrrolidinone, a vinyl acetate copolymer, a polyethylene oxide, a polyvinyl alcohol, and a combination comprising at least one of the foregoing materials.

Suitable acrylic polymers for use as an additional release-retarding material include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, or a combination comprising at least one of the foregoing polymers. The acrylic polymer may comprise methacrylate copolymers described in NF XXIV as fully polymerized copolymers of acrylic and methacrylic acid esters with a low content of quaternary ammonium groups.

Suitable alkylcelluloses and substituted alkyl celluloses include, for example, methyl cellulose, ethylcellulose, hydroxy or carboxy substituted alkyl celluloses (e.g., hydroxylpropylcellulose, crosslinked hydroxypropylcellulose, carboxymethylcellulose, crosslinked sodium carboxymethylcellulose), hydroxy substituted alkyl-alkyl celluloses (e.g., hydroxypropylmethylcellulose), or a combination comprising at least one of the foregoing alkyl celluloses.

The additional release-retarding material is present in the core in an amount of 0 to about 65 wt. % based on the total weight of the core, specifically about 0.1 to about 50 wt. %, more specifically about 10 to about 45 wt. %, and yet more specifically about 15 to about 30 wt. %. Besides the additional release-retarding material, the additional core excipients optionally includes binders, fillers, disintegrants, lubricants, glidants, and the like.

The optional disintegrant is used to facilitate the breakdown of the core in a fluid environment, specifically aqueous environments. The choice and amount of disintegrant is tailored to ensure the desired dissolution profile of the formulation or to provide the desired controlled-release in vivo. Exemplary disintegrants include a material that possesses the ability to swell or expand upon exposure to a fluid environment, especially an aqueous environment. Exemplary disintegrants include hydroxyl substituted alkyl celluloses (e.g., hydroxypropyl cellulose), starch, pregelatinized starch (e.g., Starch 1500® available from Colorcon); cross-linked sodium carboxymethylcellulose (e.g., “croscarmellose sodium”, i.e., Ac-Di-Sol® available from FMC BioPolymer of Philadelphia, Pa.); crosslinked homopolymer of N-vinyl-2-pyrrolidone (e.g., “crospovidone”, e.g., Polyplasdone® XL, Polyplasdone® XL-10, and Polyplasdone® INF-10 available from International Specialty Products, Wayne N.J.); modified starches, such as sodium carboxymethyl starch, sodium starch glycolate (e.g., Primogel®), and the like; alginates; or a combination comprising at least one of the foregoing disintegrants.

The amount of disintegrant used depends upon the disintegrant or disintegrant combination chosen and the targeted release profile of the resulting formulation. Exemplary amounts include about 0 to about 10 wt. % based on the total weight of the core, specifically about 0.5 to about 7.0 wt. %, and yet more specifically about 0.1 to about 5.0 wt. %.

Exemplary lubricants include stearates (e.g., calcium stearate, magnesium stearate, and zinc stearate), sodium stearyl fumarate, mineral oil, talc, or a combination comprising at least one of the foregoing. Glidants include, for example, silicon dioxide (e.g., fumed or colloidal). It is recognized that certain materials can function both as a glidant and a lubricant.

The lubricant or glidant is used in amounts of about 0.1 to about 15 wt. % of the total weight of the core; specifically about 0.5 to about 5 wt. %; and yet more specifically about 0.75 to about 3 wt. %.

The cores are prepared by processes known in the art, including granulation (dry or wet) and compression, spheronization, melt extrusion, hot fusion, and the like.

Once the core is formed, it is coated with an extended-release coating. The extended-release coating that substantially surrounds the core comprises a release-retarding coating material and optional other components, such as plasticizers, pore formers, and the like.

“Substantially surrounding the core” means the coating covers more than 90% of the surface area of the core, specifically more than 95%, more specifically more than 98%, and yet more specifically more than 99%.

The extended-release coating is present in the formulation at about 0.1 to about 30 wt. % based on the total weight of the core and extended-release coating, specifically about 1.0 to about 25 wt. %, more specifically about 3.0 to about 20 wt. %, yet more specifically about 4.0 to about 15 wt. %, and still yet more specifically about 5 to about 10 wt. %.

The extended-release coating is provided on the core using known coating processes such as simple or complex coacervation, interfacial polymerization, liquid drying, thermal and ionic gelation, spray drying, spray chilling, fluidized bed coating, pan coating, electrostatic deposition, compression coating, dry polymer powder coating, and the like.

The release-retarding coating material is, for example, in the form of a film coating comprising a dispersion of a hydrophobic polymer. Solvents used for application of the controlled-release coating include pharmaceutically acceptable solvents, such as water, methanol, ethanol, methylene chloride, and a combination comprising at least one of the foregoing solvents.

The extended-release profile of the active agent (either in vivo or in vitro) can be altered, for example, by using more than one release-retarding coating material, varying the thickness of the release-retarding coating material, changing the particular release-retarding coating material used, altering the relative amounts of release-retarding coating material, use of a plasticizer, altering the manner in which the plasticizer is added (e.g., when the extended-release coating is derived from an aqueous dispersion of hydrophobic polymer), by varying the amount of plasticizer relative to release-retarding coating material, by the inclusion of an additional coating excipient, by altering the method of manufacture, and the like.

Exemplary release-retarding coating materials include film-forming polymers such as an alkylcellulose including methylcellulose or ethylcellulose, a hydroxyalkylcellulose such as hydroxymethylcellulose, hydroxyethylcellulose, hydroxypropylcellulose and hydroxybutylcellulose, a hydroxyalkyl alkylcellulose such as hydroxyethyl methylcellulose and hydroxypropyl methylcellulose, a carboxyalkylcellulose such as carboxymethylcellulose, an alkali metal salt of carboxyalkylcelluloses such as sodium carboxymethylcellulose, a carboxyalkyl alkylcellulose such as carboxymethyl ethylcellulose, a carboxyalkylcellulose ester, a starch, a pectin such as sodium carboxymethylamylopectine, a chitin derivate such as chitosan, a polysaccharide such as alginic acid, alkali metal and ammonium salts thereof, a carrageenan, a galactomannan, traganth, agar-agar, gum arabicum, guar gum and xanthan gum, a polyacrylic acid and the salts thereof, a polymethacrylic acid and the salts thereof, a methacrylate copolymer, a polyvinylalcohol, a polyvinylpyrrolidone, a copolymer of polyvinylpyrrolidone with vinyl acetate, a polyalkylene oxide such as polyethylene oxide and polypropylene oxide and a copolymer of ethylene oxide and propylene oxide, or a combination comprising at least one of the foregoing materials.

The controlled-release coating optionally comprises a plasticizer, an additional film-former, a pore former, or a combination comprising at least one of the foregoing materials. In one embodiment, the release-retarding coating material comprises a plasticizer or a combination of plasticizers in an amount to substantially prevent cracking of the coating during typical use and handling. Exemplary plasticizers include fatty acids and medium-chain triglycerides (e.g., fractionated coconut oil), polyethylene glycol, dibutyl sebacate, diethyl phthalate, triethyl citrate, tributyl citrate, triacetin, acetylated monoglycerides, phthalate esters, castor oil, and the like, or a combination comprising one or more of the foregoing plasticizers. Exemplary amounts of plasticizer to substantially prevent cracking of the coating can be about 0.1 to about 40% wt based on the weight of the coating after drying, specifically about 1.0 to about 30, more specifically about 2.0 to about 25% wt. In a manufacturing setting, a rate of defective coating can be measured by the percentage of rejections that occur during the manufacturing process. An acceptable rejection rate, for example, may be set for not more than about 15% of the total quantity of the batch, more specifically not more than about 12% of the total quantity of the batch.

The formulations optionally further comprises a non-functional coating. By “functional coating” is meant to include a coating that modifies the release properties of the total formulation, for example, a controlled-release coating that provides sustained-release. By “non-functional coating” is meant to include a coating that does not significantly modify the release properties of the total formulation, for example, a cosmetic coating or an interlayer coating used to separate a functional coating from other components of the formulation. A non-functional coating can have some impact on the release of the active agent due to the initial dissolution, hydration, perforation of the coating, and the like, but would not be considered to be a significant deviation from the non-coated composition.

A pore forming material is optionally be added to the controlled-release coating to promote release of the active agent from the core. The pore forming material is organic or inorganic; it is a material that can be dissolved, extracted or leached from the coating in the environment of use; or it can have a pH-dependent solubility property; and the like. Exemplary pore forming materials include hydrophilic polymers such as a hydroxy alkyl-alkyl cellulose (e.g., hydroxypropylmethyl cellulose, and the like), a hydroxyl alkyl cellulose (e.g., hydroxypropylcellulose, and the like), or a povidone; a saccharide (e.g., lactose, and the like); a metal stearate; an inorganic salt (e.g., dibasic calcium phosphate, sodium chloride, and the like); a polyethylene glycol (e.g., polyethylene glycol (PEG) 1450, and the like); a sugar alcohol (e.g., sorbitol, mannitol, and the like); an alkali alkyl sulfate (e.g., sodium lauryl sulfate); a polyoxyethylene sorbitan fatty acid ester (e.g., polysorbate); methyacrylate copolymers (e.g., EUDRAGIT® RL); or a combination comprising at least one of the foregoing pore forming materials.

A specific release-retarding coating material includes ethyl cellulose and optionally in combination with hydroxypropymethyl cellulose.

In one embodiment, the ratio of ethyl cellulose to hydroxypropylmethyl cellulose is about 90:10, specifically about 60:40, and yet more specifically about 50:50.

In another embodiment, the ratio of ethyl cellulose to hydroxypropylmethyl cellulose is about 1:1, specifically about 1:2, more specifically about 1:3, and yet more specifically about 1:5.

In one embodiment, the controlled-release core releases the core active agent over a period of about 6 hours to about 24 hours, specifically about 12 hours or about 24 hours.

In one embodiment, the formulation comprises a controlled-release portion in the form of a coated core and an immediate-release portion disposed on at least a portion of the controlled-release core. The formulation is prepared, for example, as a bilayer tablet, a coated tablet, a compression-coated tablet, or any other suitable form. In one embodiment, the immediate-release portion is in the form of a coating substantially surrounding the coated core applied, for example, using spray coating, compression coating, or other suitable technique. The active agent present in the controlled-release portion and the immediate-release portion can be the same or different.

In one embodiment, the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer, wherein the formulation exhibits a dissolution profile such that at one hour after combining the formulation with 900 ml of deionized water at 37° C.±0.5° C. according to USP 28 <711> test method 2 (paddle), 75 rpm paddle speed, about 10 to about 55 wt. % of the total amount of active agent is released.

In another embodiment, the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer, wherein the formulation exhibits a dissolution profile such that two hours after combining the formulation with 900 ml of deionized water at 37° C.±0.5° C. according to USP 28 <711> test method 2 (paddle), 75 rpm paddle speed, about 20 to about 65 wt. % of the total amount of the active agent is released.

In yet another embodiment, the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer, wherein the formulation exhibits a dissolution profile such that four hours after combining the formulation with 900 ml of deionized water at 37° C.±0.5° C. according to USP 28 <711> test method 2 (paddle), 75 rpm paddle speed, about 35 to about 85 wt. % of the total amount of the active agent is released.

In still another embodiment, the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer, wherein the formulation exhibits a dissolution profile such that eight hours after combining the formulation with 900 ml of deionized water at 37° C.±0.5° C. according to USP 28 <711> test method 2 (paddle), 75 rpm paddle speed, about 50 to about 100 wt. % of the total amount of the active agent is released.

In another embodiment, the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer, wherein the formulation exhibits a dissolution profile such that twelve hours after combining the formulation with 900 ml of deionized water at 37° C.±0.5° C. according to USP 28 <711> test method 2 (paddle), 75 rpm paddle speed, about 50 to about 100 wt. % of the total amount of the active agent is released.

Also included herein are pharmaceutical kits which comprise one or more containers containing a controlled-release formulation, wherein the formulation comprises a core comprising an active agent and a wax excipient; and an extended-release coating substantially surrounding the core comprising a release-retarding coating material. The kits may further comprise one or more conventional pharmaceutical kit components, such as, for example, one or more containers to aid in facilitating compliance with a particular dosage regimen; one or more carriers; printed instructions, either as inserts or as labels, indicating quantities of the components to be administered, or guidelines for administration. Exemplary kits can be in the form of bubble or blister pack cards, optionally arranged in a desired order for a particular dosing regimen. Suitable blister packs that can be arranged in a variety of configurations to accommodate a particular dosing regimen are well known in the art or easily ascertained by one of ordinary skill in the art.

In one embodiment, a method of treating a patient comprises administering a controlled release levetiracetam formulation to a patient in need thereof, wherein the controlled-release formulation comprises a core comprising levetiracetam or a pharmaceutically acceptable salt, solvate, hydrate, crystalline form, or non-crystalline form thereof, and a wax excipient, wherein the core is substantially free of a non-wax binder; and an extended-release coating substantially surrounding the core comprising a release-retarding coating material and a plasticizer. The patient may be treated for epilepsy, neuropathic pain, seizures, and the like.

### EXAMPLES

### Example 1

### Levetiracetam Extended-Release Tablets, 750 mg

An extended-release levetiracetam tablet wax core is prepared having the components listed in Table 1a below.

TABLE 1a

| Component | Weight (mg/tablet) | % weight of tablet core |
|---|---|---|
| Levetiracetam | 750.0 | 75 |
| Carnauba Wax | 150.0 | 15 |
| Stearic acid | 85.0 | 8.5 |
| Silicon dioxide | 7.5 | 0.75 |
| (Syloid 244 FP) | | |
| Magnesium Stearate | 7.5 | 0.75 |
| Total core tablet weight | 1000 | 100 |

The tablet cores are prepared by granulating levetiracetam and carnauba wax in a mixer/granulator for five minutes. Stearic acid is dissolved in denatured alcohol with mixing and gentle heat. The stearic acid mixture is added to the active agent/wax mixture and granulated to form granules. The resulting granules are dried and milled. The milled granules are charged to a Gemco Blender to which screened silicon dioxide is added and mixed. Screened magnesium stearate is then added and mixed to form a blend. The resulting blend is then compressed into tablet cores to a hardness of 12-18 kp, specifically 15 kp.

A reference extended-release levetiracetam tablet core is prepared having the components listed in Table 1b below.

TABLE 1b

| Component | Weight (mg/tablet) |
|---|---|
| Part I | |
| Levetiracetam | 750.0 |
| Povidone (Plasdone K-90) | 10.0 |
| Part II | |
| Hypromellose (Methocel | 250.0 |
| K100M Premium CR) | |
| Hypromellose (Methocel | 41.0 |
| K100 Premium LV CR) | |
| Talc | 6.0 |
| Silicon dioxide (Syloid 244 FP) | 6.0 |
| Magnesium stearate | 12.0 |
| Total core tablet weight | 1075 |

The reference tablet core is prepared by wet granulating levetiracetam and povidone with water. The formed granules are dried. The dried granules are blended with two types of hypromellose and sized. The sized granules are blended with silicon dioxide followed by magnesium stearate and talc and the final mixture is compressed into tablet cores to a hardness of 10-20 kp, specifically 15 kp.

The tablet cores are then coated with a coating solution to achieve a targeted weight gain to form coated, extended-release levetiracetam tablets. The components of the coatings are listed in Table 2 below; Formulations A, B, and C contain the wax cores from Table 1a and Reference Formulation D contains the hypromellose cores described in Table 1b.

TABLE 2

| | | | |
|---|---|---|---|
| A | B | C | D |

| | |
|---|---|
| Component | Weight (mg/tablet) |

| | | | | |
|---|---|---|---|---|
| Tablet Cores | 1000 | 1000 | 1000 | 1075 |
| Surelease | 30.4 | 20 | 36 | — |
| (E-7-19010) | | | | |
| [Ethylcellulose- | | | | |
| based coating] | | | | |
| Opadry clear | 49.6 | 60 | 44 | — |
| (YS-3-7011) | | | | |
| [Hypromellose- | | | | |
| based coating] | | | | |
| Ethylcellulose | — | — | — | 19.565 |
| aqueous dispersion | | | | |
| (Aquacoat ECD-30) | | | | |
| Polyethylene glycol | — | — | — | 7.525 |
| 400 | | | | |
| Hypromellose | — | — | — | 10.535 |
| (Methocel E3 | | | | |
| Premium LV) | | | | |
| Opadry purple | — | 22 | — | — |
| Opadry II Pink | — | — | 22 | — |
| Total tablet weight | 1080 | 1102 | 1102 | 1112.625 |

In a generalized coating process for Formulations A, B, and C, Opadry clear is dissolved in water and added to a container containing Surelease with mixing to form a coating solution. The coating solution is applied to the wax tablet cores using a tablet spray coater. The coated tablets are dried in an oven at 40° C. for forty-eight hours. Formulations B and C are further coated with a color coating for identification purposes.

In a generalized coating process for Reference Formulation D, hypromellose and polyethylene glycol are added to purified water with stirring until a clear solution is obtained. This solution is added to the ethylcellulose aqueous dispersion with stirring. The coating solution is applied to the hypromellose tablet cores using a tablet spray coater. The coated tablets are dried in an oven at 40° C. for forty-eight hours.

### Example 2

### Comparative Dissolution

A comparison of in vitro dissolution was conducted between the extended-release tablet core (wax); the coated, extended-release levetiracetam tablets of Formulations A, B, and C; and Reference Formulation D, all of Example 1. The test method protocol according to USP 26, 711, 900 milliliters of deionized water at 37° C.±0.5° C. and a paddle speed of 75 rotations per minute (rpm) is used. The results of the dissolution analyses are summarized in Table 3; each data point is an average of six samples.

TABLE 3

| | Example 8 | Example 8 | Example 8 | Example 8 | Example 8 |
|---|---|---|---|---|---|
| | A | B | C | Wax core, | Reference |
| Time | (coated) | (coated) | (coated) | uncoated | D (coated) |
| (hr) | % dissolved | | | | |
| 0 | 0 | 0 | 0 | 0 | 0 |
| 1 | 28 | 35 | 18 | 41 | 26 |
| 2 | 43 | 52 | 29 | 54 | 40 |
| 4 | 62 | 71 | 43 | 72 | 58 |
| 8 | 83 | 90 | 62 | 89 | 81 |
| 12 | 94 | 100 | 75 | 98 | 92 |
| 18 | 99 | 103 | 88 | 101 | 98 |
| 24 | 100 | 104 | 95 | 103 | 100 |

As the dissolution results in Table 3 indicate, the coated extended-release levetiracetam tablets provide a long, controlled delivery of levetiracetam.

### Example 3

### Relative Bioavailability Under Fasting Conditions of the Extended-Release Coated Tablet Formulations of Example 1 (Wax) Versus Reference Extended-Release Coated Tablet (Hypromellose)

A 4-arm, open-label, single-dose, randomized, four-period, four-treatment, fasted relative bioavailability study of the coated extended-release levetiracetam Formulations A-C of Example 1 versus reference formulation D (“Reference”) of Example 1 was performed in healthy, adult volunteers. The study was performed on 20 subjects. Each subject participates in four dosing periods separated by a washout period of at least seven days. The four dosing regimens on four separate occasions are one 750 mg Formulation A tablet (test product A, Formulation A of Example 1), one 750 mg Formulation B tablet (test product B,

TABLE 4
Formulation A, Fasting

| | | | | |
|---|---|---|---|---|
| | | | | 90% Confidence |
| | | | | Interval |
| | Formulation | Reference | % | (Lower Limit, |
| PK variable | A | D | Ratio | Upper Limit) |

Ln-transformed data
Geometric Mean

| | | | | |
|---|---|---|---|---|
| Cₘₐₓ (ng/ml) | 8.22 | 9.08 | 90.47 | (85.41, 95.83) |
| AUC₀₋ₜ | 155.56 | 166.99 | 93.16 | (86.42, 100.43) |
| (ng-hr/ml) | | | | |
| AUC_{0-INF} | 167.02 | 178.22 | 93.17 | (86.86, 101.11) |
| (ng-hr/ml) | | | | |

Non-transformed data
least squares mean

| | | | | |
|---|---|---|---|---|
| Cₘₐₓ (ng/ml) | 8.36 | 9.36 | 89.25 | (82.97, 95.54) |
| AUC₀₋ₜ | 161.40 | 171.78 | 93.96 | (88.27, 99.65) |
| (ng-hr/ml) | | | | |
| AUC_{0-INF} | 172.35 | 182.66 | 94.35 | (87.85, 100.85) |
| (ng-hr/ml) | | | | |
| Tₘₐₓ | 4.63 | 4.48 | 103.46 | (87.18, 119.75) |
| kₑ | 0.08 | 0.08 | 99.43 | (93.28, 105.59) |
| t_{1/2} | 8.71 | 8.51 | 102.28 | (87.18, 117.38) |

Formulation B of Example 1), one 750 mg Formulation C tablet (test product C, Formulation C of Example 1), and one 750 mg Reference tablet (Reference D of Example 1) preceded by an overnight fast of at least 10 hours. Subjects will be confined at the early evening prior to and until at least 24 hours after dosing. Blood samples will be drawn from each subject for drug content analysis at time zero (predose) and after dose administration every ½ hour for the first eight hours, then at hours 9, 10, 12, 16, 20, 24, 36, 48, and 72. Levetiracetam plasma concentrations in the blood samples will be measured using a validated bioanalytical method.

The levetiracetam concentration-time data will be used to calculate the following pharmacokinetic parameters: AUC₀₋ₜ, AUC_{0-∞}, Cₘₐₓ, Tₘₐₓ, kₑ, and t_{1/2}. The pharmacokinetic parameters will be evaluated statistically by an analysis of variance (ANOVA) appropriate for the experimental design of the study. Analyses for AUC₀₋ₜ, AUC_{0-∞}, and Cₘₐₓ will be performed on ln-transformed data. For ln-transformed AUC₀₋ₜ, AUC_{0-∞}, and Cₘₐₓ, estimates of the adjusted differences between treatment means and the standard error associated with these differences will be used to construct a 90% confidence interval for the ratio of the test to reference population means. The results are provided in Tables 4, 5, and 6 below.

TABLE 5
Formulation B, Fasting

| | | | | |
|---|---|---|---|---|
| | | | | 90% Confidence |
| | | | | Interval |
| | Formulation | Reference | % | (Lower Limit, |
| PK variable | B | D | Ratio | Upper Limit) |

Ln-transformed data
Geometric Mean

| | | | | |
|---|---|---|---|---|
| Cₘₐₓ (ng/ml) | 10.15 | 9.08 | 111.69 | (105.44, 118.3) |
| AUC₀₋ₜ | 166.59 | 166.99 | 99.76 | (92.54, 107.55) |
| (ng-hr/ml) | | | | |
| AUC_{0-INF} | 178.02 | 178.22 | 99.89 | (92.58, 107.77) |
| (ng-hr/ml) | | | | |

Non-transformed data
least squares mean

| | | | | |
|---|---|---|---|---|
| Cₘₐₓ (ng/ml) | 10.43 | 9.36 | 111.34 | (105.06, 117.63) |
| AUC₀₋ₜ | 168.95 | 171.78 | 98.35 | (92.66, 104.04) |
| (ng-hr/ml) | | | | |
| AUC_{0-INF} | 180.51 | 182.66 | 98.82 | (92.32, 105.32) |
| (ng-hr/ml) | | | | |
| Tₘₐₓ | 4.30 | 4.48 | 96.09 | (79.8, 112.38) |
| kₑ | 0.08 | 0.08 | 100.59 | (94.44, 106.75) |
| t_{1/2} | 8.54 | 8.51 | 100.35 | (85.25, 115.45) |

TABLE 6
Formulation C, Fasting

| | | | | |
|---|---|---|---|---|
| | | | | 90% Confidence |
| | | | | Interval |
| | Formulation | Reference | % | (Lower Limit, |
| PK variable | C | D | Ratio | Upper Limit) |

Ln-transformed data
Geometric Mean

| | | | | |
|---|---|---|---|---|
| Cₘₐₓ (ng/ml) | 7.28 | 9.08 | 80.17 | (75.68, 84.92) |
| AUC₀₋ₜ | 140.04 | 166.99 | 83.86 | (77.79, 90.41) |
| (ng-hr/ml) | | | | |
| AUC_{0-INF} | 157.88 | 178.22 | 88.59 | (82.1, 95.58) |
| (ng-hr/ml) | | | | |

Non-transformed data
least squares mean

| | | | | |
|---|---|---|---|---|
| Cₘₐₓ (ng/ml) | 7.45 | 9.36 | 79.53 | (73.25, 85.81) |
| AUC₀₋ₜ | 146.50 | 171.78 | 85.29 | (79.6, 90.98) |
| (ng-hr/ml) | | | | |
| AUC_{0-INF} | 165.65 | 182.66 | 90.69 | (84.19, 97.19) |
| (ng-hr/ml) | | | | |
| Tₘₐₓ | 5.68 | 4.48 | 126.82 | (110.53, 143.1) |
| kₑ | 0.08 | 0.08 | 93.06 | (86.91, 99.22) |
| t_{1/2} | 10.21 | 8.51 | 119.87 | (104.77, 134.97) |

As shown by the results, by varying the coating type on the tablets (ratio of ethylcellulose to hydroxypropylmethyl celluse, and coating weight), it is possible to control the pharmacokinetic profile of the dosage form to obtain a desired release profile in vivo.

### Example 4

### Relative Bioavailability Under Non-Fasting Conditions of the Extended-Release Coated Tablet Formulations of Example 1 (Wax) Versus Reference Extended-Release Coated Tablet (Hypromellose)

A 4-arm, open-label, single-dose, randomized, four period, four treatment, non-fasted relative bioavailability study of the coated extended-release levetiracetam Formulations A-C of Example 1 versus reference formulation D (“Reference”) of Example 1 was performed in healthy, adult volunteers. The study was performed on 19 subjects. Each subject participates in four dosing periods separated by a washout period of at least seven days. The four dosing regimens on four separate occasions are one 750 mg Formulation A tablet (test product A, Formulation A of Example 1), one 750 mg Formulation B tablet (test product B, Formulation B of Example 1), one 750 mg Formulation C tablet (test product C, Formulation C of Example 1), and one 750 mg Reference tablet (Reference D of Example 1) administered within five minutes of consuming an entire standard high-fat breakfast. Subjects will be confined at the early evening prior to and until at least 24 hours after dosing. Blood samples will be drawn from each subject for drug content analysis at time zero (predose) and after dose administration every ½ hour for the first eight hours, then at hours 9, 10, 12, 16, 20, 24, 36, 48, and 72. Levetiracetam plasma concentrations in the blood samples will be measured using a validated bioanalytical method.

The levetiracetam concentration-time data will be used to calculate the following pharmacokinetic parameters: AUC₀₋ₜ, AUC_{0-∞}, Cₘₐₓ, Tₘₐₓ kₑ, and t_{1/2}. The pharmacokinetic parameters will be evaluated statistically by an analysis of variance (ANOVA) appropriate for the experimental design of the study. Analyses for AUC₀₋ₜ, AUC_{0-∞}, and Cₘₐₓ will be performed on ln-transformed data. For ln-transformed AUC₀₋ₜ, AUC_{0-∞}, and Cₘₐₓ, estimates of the adjusted differences between treatment means and the standard error associated with these differences will be used to construct a 90% confidence interval for the ratio of the test to reference population means. The results are provided in Tables 7, 8, and 9 below.

TABLE 7
Formulation A, Non-Fasting

| | | | | |
|---|---|---|---|---|
| | | | | 90% Confidence |
| | | | | Interval |
| | Formulation | Reference | % | (Lower Limit, |
| PK variable | A | D | Ratio | Upper Limit) |

Ln-transformed data
Geometric Mean

| | | | | |
|---|---|---|---|---|
| Cₘₐₓ (ng/ml) | 9.09 | 10.67 | 85.21 | (80.71, 89.96) |
| AUC₀₋ₜ | 182.43 | 183.09 | 99.64 | (94.78, 104.75) |
| (ng-hr/ml) | | | | |
| AUC_{0-INF} | 193.88 | 193.57 | 100.16 | (95.85, 104.66) |
| (ng-hr/ml) | | | | |

Non-transformed data
least squares mean

| | | | | |
|---|---|---|---|---|
| Cₘₐₓ (ng/ml) | 9.28 | 10.80 | 85.96 | (81.37, 90.54) |
| AUC₀₋ₜ | 185.77 | 186.65 | 99.53 | (95.79, 103.26) |
| (ng-hr/ml) | | | | |
| AUC_{0-INF} | 197.03 | 196.75 | 100.14 | (96.58, 103.7) |
| (ng-hr/ml) | | | | |
| Tₘₐₓ | 6.42 | 6.31 | 101.76 | (87.99, 115.54) |
| kₑ | 0.0842 | 0.0875 | 96.17 | (90.26, 102.07) |
| t_{1/2} | 8.42 | 8.04 | 104.71 | (97.05, 112.37) |

TABLE 8
Formulation B, Non-Fasting

| | | | | |
|---|---|---|---|---|
| | | | | 90% Confidence |
| | | | | Interval |
| | Formulation | Reference | % | (Lower Limit, |
| PK variable | B | D | Ratio | Upper Limit) |

Ln-transformed data
Geometric Mean

| | | | | |
|---|---|---|---|---|
| Cₘₐₓ (ng/ml) | 10.96 | 10.67 | 102.73 | (97.31, 108.46) |
| AUC₀₋ₜ | 182.68 | 183.09 | 99.78 | (94.91, 104.89) |
| (ng-hr/ml) | | | | |
| AUC_{0-INF} | 196.11 | 193.57 | 101.31 | (96.96, 105.87) |
| (ng-hr/ml) | | | | |

Non-transformed data
least squares mean

| | | | | |
|---|---|---|---|---|
| Cₘₐₓ (ng/ml) | 11.11 | 10.80 | 102.91 | (98.33, 107.49) |
| AUC₀₋ₜ | 186.01 | 186.65 | 99.66 | (95.92, 103.4) |
| (ng-hr/ml) | | | | |
| AUC_{0-INF} | 198.72 | 196.75 | 101.00 | (97.44, 104.56) |
| (ng-hr/ml) | | | | |
| Tₘₐₓ | 5.33 | 6.31 | 84.49 | (70.71, 98.26) |
| kₑ | 0.0834 | 0.0875 | 95.31 | (89.4, 101.21) |
| t_{1/2} | 8.50 | 8.04 | 105.74 | (98.08, 113.4) |

TABLE 9
Formulation C, Non-Fasting

| | | | | |
|---|---|---|---|---|
| | | | | 90% Confidence |
| | | | | Interval |
| | Formulation | Reference | % | (Lower Limit, |
| PK variable | C | D | Ratio | Upper Limit) |

Ln-transformed data
Geometric Mean

| | | | | |
|---|---|---|---|---|
| Cₘₐₓ (ng/ml) | 8.16 | 10.67 | 76.47 | (72.43, 80.73) |
| AUC₀₋ₜ | 164.97 | 183.09 | 90.11 | (85.71, 94.72) |
| (ng-hr/ml) | | | | |
| AUC_{0-INF} | 178.93 | 193.57 | 92.44 | (88.46, 96.59) |
| (ng-hr/ml) | | | | |

Non-transformed data
least squares mean

| | | | | |
|---|---|---|---|---|
| Cₘₐₓ (ng/ml) | 8.37 | 10.80 | 77.52 | (72.94, 82.1) |
| AUC₀₋ₜ | 171.55 | 186.65 | 91.91 | (88.17, 95.65) |
| (ng-hr/ml) | | | | |
| AUC_{0-INF} | 185.04 | 196.75 | 94.05 | (90.49, 97.61) |
| (ng-hr/ml) | | | | |
| Tₘₐₓ | 6.79 | 6.31 | 107.59 | (93.82, 121.37) |
| kₑ | 0.0797 | 0.0875 | 91.04 | (85.13, 96.94) |
| t_{1/2} | 8.87 | 8.04 | 110.38 | (102.72, 118.03) |

As shown by the results, by varying the coating type on the tablets (ratio of ethylcellulose to hydroxypropylmethyl celluse, and coating weight), it is possible to control the pharmacokinetic profile of the dosage form to obtain a desired release profile in vivo. Also, it is shown that it is possible to achieve equivalence in the non-fasting state to the fasting state for Cₘₐₓ and AUC. Comparing the results of the in vivo study in the fasting state in Example 3 to the results of the in vivo study in the non-fasting state in Example 4, one of skill in the art can interpret the results to mean the formulations exhibit no significant food effect.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A controlled-release formulation, comprising:
a core comprising
levetiracetam or a pharmaceutically acceptable salt, solvate, hydrate, crystalline form, or non-crystalline form thereof, and
a wax excipient; and
an extended-release coating substantially surrounding the core comprising a release-retarding coating material and a plasticizer.

2. The formulation of claim 1, wherein the wax excipient is carnauba wax, vegetable wax, fruit wax, microcrystalline wax, bees wax, hydrocarbon wax, paraffin wax, cetyl esters wax, non-ionic emulsifying wax, anionic emulsifying wax, candelilla wax, stearyl alcohol, cetyl alcohol, cetostearyl alcohol, lauryl alcohol, myristyl alcohol, a hydrogenated vegetable oil, a hydrogenated castor oil, a fatty acid, a fatty acid ester, a fatty acid glyceride, a polyethylene glycol having a Mₙ, of greater than about 3000, or a combination comprising at least one of the foregoing wax excipients.

3. The formulation of claim 1, wherein the wax excipient is a combination of carnauba wax and stearic acid.

4. The formulation of claim 1, wherein the wax excipient is present in an amount of about 10 to about 40 wt. % based on the total weight of the core.

5. The formulation of claim 1, wherein the wax excipient is present in an amount of about 20 to about 30 wt. % based on the total weight of the core.

6. The formulation of claim 1, wherein the core is prepared by wet granulation and compression processes.

7. The formulation of claim 1, wherein the core further comprises a glidant, a lubricant, or a combination comprising at least one of the foregoing.

8. The formulation of claim 1, wherein the release-retarding coating material comprises a film forming polymer, wherein the film forming polymer is an alkylcellulose, a carboxyalkylcellulose, an alkali metal salt of a carboxyalkylcellulose, a carboxyalkyl alkylcellulose, a carboxyalkylcellulose ester, a starch, a pectin, a chitine derivate, a polysaccharide, a carrageenan, a galactomannas, traganth, agar-agar, gum arabicum, guar gum, xanthan gum, a polyacrylic acid, a polymethacrylic acid, a methacrylate copolymer, a polyvinyl alcohol, a copolymer of polyvinylpyrrolidone with vinyl acetate, a polyalkylene oxide, a copolymer of ethylene oxide and propylene oxide, or a combination comprising at least one of the foregoing film forming polymers.

9. The formulation of claim 8, wherein the film forming polymer is ethylcellulose, carboxymethyl cellulose, sodium carboxymethylcellulose, carboxymethyl ethylcellulose, carboxymethylcellulose ester, sodium carboxymethylamylopectine, chitosan, alginic acid, alkali metal salt of alginic acid, ammonium salt of alginic acid, or a combination comprising at least one of the foregoing film forming polymers.

10. The formulation of claim 8, wherein the film forming polymer is ethylcellulose.

11. The formulation of claim 1, wherein the extended-release coating further comprises a pore former, wherein the pore former is a hydrophilic polymer, a hydroxy alkyl-alkyl cellulose, a hydroxyl alkyl cellulose, a povidone, a saccharide, an inorganic salt, a sugar alcohol, a polyoxyethylene sorbitan fatty acid ester, a hydrophilic methyacrylate copolymer, or a combination comprising at least one of the foregoing pore formers.

12. The formulation of claim 11, wherein the pore former is a hydroxypropylmethyl cellulose.

13. The formulation of claim 1, wherein the extended-release coating is present at about 1.0 to about 20 wt. % based on the total weight of the core and extended-release coating.

14. The formulation of claim 1, wherein the extended-release coating is present at about 5.0 to about 10 wt. % based on the total weight of the core and extended-release coating.

15. The formulation of claim 1, wherein the formulation is bioequivalent to a reference drug product according to New Drug Application No. 022285 when administered to a patient in a fasted state.

16. The formulation of claim 1, wherein the formulation exhibits
a ratio of a geometric mean of logarithmic transformed AUC_{0-∞} of the formulation to a geometric mean of logarithmic transformed AUC_{0-∞} of reference drug (New Drug Application No. 022285) of about 0.80 to about 1.25;
a ratio of a geometric mean of logarithmic transformed AUC₀₋ₜ of the formulation to a geometric mean of logarithmic transformed AUC₀₋ₜ of reference drug (New Drug Application No. 022285) of about 0.80 to about 1.25;
a ratio of a geometric mean of logarithmic transformed Cₘₐₓ of the formulation to a geometric mean of logarithmic transformed Cₘₐₓ of reference drug (New Drug Application No. 022285) of about 0.70 to about 1.43; or
a ratio of a geometric mean of logarithmic transformed Cₘₐₓ of the formulation to a geometric mean of logarithmic transformed Cₘₐₓ of reference drug (New Drug Application No. 022285) of about 0.80 to about 1.25,
wherein the foregoing are determined under fasting conditions.

17. The formulation of claim 1, wherein the formulation exhibits a dissolution profile such that at one hour after combining the formulation with 900 ml of deionized water at 37° C.±0.5° C. when tested using a tablet dissolution apparatus equipped with a paddle stirring element, 75 rpm paddle speed, about 10 to about 55 wt. % of the total amount of active agent is released.

18. The formulation of claim 17, wherein after two hours, about 20 to about 65 wt. % of the total amount of the active agent is released.

19. The formulation of claim 18, wherein after four hours, about 35 to about 85 wt. % of the total amount of the active agent is released.

20. The formulation of claim 19, wherein after eight hours about 50 to about 100 wt. % of the total amount of the active agent is released.

21. The formulation of claim 20, wherein after twelve hours about 60 to about 100 wt. % of the total amount of the active agent is released.

22. The formulation of claim 1, wherein the core is substantially free of a non-wax binder

23. A controlled-release formulation, comprising:
a core consisting essentially of
levetiracetam or a pharmaceutically acceptable salt, solvate, hydrate, crystalline form, or non-crystalline form thereof, and
a wax excipient; and
an extended-release coating substantially surrounding the core comprising a release-retarding coating material and a plasticizer;
wherein the formulation exhibits substantially no food effect.

24. A method of treating a patient, comprising
administering the formulation of claim 1 to a patient.
